(19) United States
(12) Patent Application Publication
(10) Pub. No.: US 2018/0355130 A1
(43) Pub. Date: Dec. 13, 2018

(54) THERMOPLASTIC FILM AND AN ELASTIC COMPOSITE MADE WITH THE SAME

(71) Applicant: H. B. Fuller Computer, St. Paul, MN (US)

(72) Inventors: Ameara S. Mansour, Woodbury, MN (US); Kevin P. Davis, Woodbury, MN (US); Tara Misra, Shoreview, MN (US); Kristy J. Beckman, Dellwood, MN (US); Kirstin A. Hedin, Ham Lake, MN (US)

(21) Appl. No.: 16/002,709

(22) Filed: Jun. 7, 2018

Related U.S. Application Data
(60) Provisional application No. 62/516,403, filed on Jun. 7, 2017.

(51) Int. Cl. C08J 5/18; A61L 15/24; D06N 3/00; D06N 3/04; B32B 5/02; B32B 27/30; B32B 27/12; B32B 37/15

(52) U.S. Cl. CPC C08J 5/18; B32B 2535/00; D06N 3/0011; D06N 3/045; B32B 5/022; B32B 27/302; B32B 27/12; B32B 37/153; C08J 2353/02; D06N 2205/06; D06N 2209/1635; D10B 2401/061; D10B 2509/026; D10B 2509/022; B32B 2325/00; B32B 2307/51; B32B 2555/00; A61L 15/24

(57) ABSTRACT

The invention features a thermoplastic film comprising greater than 70% by weight of a styrene block copolymer having an unsaturated mid block, diblock content of no greater than about 10% by weight and a MFR of from about 15 to about 200 ASTM D 1238 (190° C., 2.16-kg). The thermoplastic film can be used to form elastic composites that are useful in disposable articles (e.g. disposable absorbent articles).

This application claims the benefit of U.S. Provisional Application No. 62/516,403 filed Jun. 7, 2017, which is incorporated herein.

### BACKGROUND

Low Melt Flow Rate (MFR), thermoplastic polymers have been used in the past to provide elasticity to various disposable articles e.g. diapers, training pants, swim wear, absorbent underpants and adult incontinence articles.

Such polymers work well but it would be useful to have a thermoplastic material that could be applied at a lower temperature while still providing similar elastic properties.

### SUMMARY

Applicants have discovered that a thermoplastic film consisting primarily or essentially of certain styrene block copolymers combines several desirable properties including manufacturing efficiency (no need for hydrogenation), high Melt Flow Rate (MFR), low odor and a desirable retractive profile (i.e. improved snap back) as demonstrated by low mechanical hysteresis values. These properties make these thermoplastic films well suited to forming elastic composites. Further, the thermoplastic film can be formed at a lower processing temperature while still providing elastic properties similar to those of lower MFR materials (which require higher application temperatures).

### DETAILED DESCRIPTION

In one aspect, the invention features a thermoplastic film comprising greater than 70% by weight of a styrene block copolymer having an unsaturated mid block, diblock content of no greater than about 10% by weight and a MFR of from about 15 to about 200 ASTM D 1238 (190° C., 2.16-kg).

In one embodiment, the thermoplastic film has a cross direction mechanical hysteresis according to the Mechanical Hysteresis test method of from 0 to about 0.70. In a different embodiment, the thermoplastic film is elastic.

In another embodiment, the thermoplastic film comprises greater than 80% by weight of the styrene block copolymer. In one embodiment, the thermoplastic film consists essentially of the styrene block copolymer. In another embodiment, the styrene block copolymer has an average styrene content of from about 25% by weight to about 40% by weight. In a different embodiment, the styrene block copolymer has an ASTM D 1238 (190° C., 2.16 kg) melt flow rate of from about 15 to about 100. In one embodiment, the styrene block copolymer is a styrene-isoprene-styrene block copolymer.

In a different embodiment, the thermoplastic film is free of a tackifying agent.

In another aspect, the invention features an elastic composite including a first substrate; and the thermoplastic film layered on top of the first substrate. In one embodiment, the first substrate is nonwoven. In another embodiment, the thermoplastic film is applied to the substrate while molten. In a different embodiment, the thermoplastic film is applied using an applicator method selected from the group consisting of slot and non-contact coating.

In one embodiment, the invention includes a disposable article comprising the elastic composite. In a different embodiment the disposable article is selected from the group consisting of diaper, adult incontinence product, feminine hygiene product and medical bandage.

In another aspect, the invention features a method of forming an elastic composite comprising the steps of applying a molten thermoplastic film to one substrate, and contacting the molten thermoplastic film to a second substrate to form an elastic composite, the thermoplastic film comprising greater than 70% by weight of a styrene block copolymer having an unsaturated mid block, diblock content of no greater than about 10% by weight and a MFR of from about 15 to about 200 ASTM D 1238 (190° C., 2.16-kg)

### DEFINITIONS

Elastic as used herein means that the layer referred to deforms when subjected to an external load and recovers, at least partially, from the deformation when the load is removed.

### Thermoplastic Film

The invention includes a thermoplastic film comprising greater than 70% by weight of a styrene block copolymer having an unsaturated mid block, diblock content of no greater than about 10% by weight and a MFR of front about 15 to about 200 ASTM D 1238 (190° C., 2.16-kg).

The thermoplastic film includes a styrene block copolymer. The styrene block copolymer can include one or more styrene block copolymers. The styrene block copolymer can make up greater than 70% by weight, greater than 75% by weight, greater than 78% by weight, greater than 80% by weight, greater than 90% by weight, or even greater than 95% by weight of the thermoplastic film. The thermoplastic film can consist essentially of the styrene block copolymer.

The thermoplastic film can include less than 10% of a tackifying agent, less than 5% of a tackifying agent, or even be essentially free of a tackifying agent.

The thermoplastic film can be light in color. The thermoplastic film can be pigmented to an opaque color such as e.g. pink, blue, white, etc.

The thermoplastic film can have a thickness of from about 10 gsm (grains per square meter) to about 200 gsm, from about 15 gsm to about 100 gsm, or even from about 20 gsm to about 75 gsm.

The thermoplastic film has elastic properties as witnessed by its mechanical hysteresis values. The thermoplastic film can have a cross direction mechanical hysteresis according to the Mechanical Hysteresis test method of from 0 to about 0.70, or even from about 0.05 to about 0.50.

### Styrene Block Copolymer

The thermoplastic film includes one or more styrene block copolymers having an unsaturated mid block.

The styrene block copolymer includes an aromatic vinyl polymer block and a conjugated diene polymer block. The blocks can be arranged in a variety of configurations including, e.g., linear, branched, radial, star and combinations thereof. The aromatic vinyl polymer block can be derived from a variety of aromatic vinyl compounds including, e.g., styrene, alpha-methylstyrene, beta-methylstyrene, o-, m-, p-methylstyrene, t-butylstyrene, 2,4,6-trimethylstyrene, monofluorostyrene, difluorostyrene, monochlorostyrene, dichlorostyrene, methoxy styrene, 1,3-vinylnaphthalene, vinylanthracene, indene, acenaphthylene, and combinations thereof. The diene polymer block can be derived from a variety of diene-containing compounds including, e.g., isoprene, butadiene, hexadiene, 2,3-dimethyl-1,3-butadiene and 1,3-pentadiene, farnesene, and combinations thereof.

Useful styrene block copolymers include, e.g., triblock, multi-arm, and radial copolymers including, e.g., styrene-butadiene-styrene (SBS), styrene-isoprene-styrene (SIS), styrene-isoprene-butadiene-styrene (SIBS), and combinations thereof.

The styrene block copolymer can have a diblock content of no greater than about 10% by weight, no greater than about 5% by weight, no greater than about 3% by weight, or even no greater than about 1% by weight.

The styrene block copolymer can have a styrene content of at least about 20% by weight, at least about 25% by weight, from about 20% to about 45% by weight, or even from about 25% to about 40% by weight.

The styrene block copolymer has an MFR ASTM D 1238 (190° C., 2.16 kg) of at least about 12, from about 12 to about 200, or even from about 15 to about 100.

If more than one styrene block copolymer is present having an MFR ASTM 1238 (190° C., 2.16 kg) of from about 12 to about 200 and a diblock content of no greater than about 10% by weight, the weight average of the SBCs present can fall within the ranges listed for diblock content, styrene content and MFR.

As an example, if the thermoplastic film includes two styrene block copolymers A and B. Polymer A is present at 25 weight % with a diblock content of 0% and polymer B is present at 25 weight % with a diblock content of 5%. The weight average diblock content of the styrene block copolymers is calculated the following way: 0.5(0)+0.5(0.05)=0.025 or 2.5% by weight.

### Tackifying Agent

The thermoplastic film can include a tackifying agent. The tackifying agent can function to tackify the mid block of the SBC or to reinforce the end block of the SBC. The tackifying agent can be fluid or solid at room temperature. Suitable classes of tackifying agents include, e.g., aromatic, aliphatic and cycloaliphatic hydrocarbon resins, mixed aromatic and aliphatic modified hydrocarbon resins, aromatic modified aliphatic hydrocarbon resins, and hydrogenated versions thereof; terpenes, modified terpenes and hydrogenated versions thereof; natural rosins, modified rosins, rosin esters, and hydrogenated versions thereof; low molecular weight polylactic acid; and combinations thereof. Examples of useful natural and modified rosins include gum rosin, wood rosin, tall oil rosin, distilled rosin, hydrogenated rosin, dimerized rosin and polymerized rosin. Examples of useful rosin esters include e.g., glycerol esters of pale wood rosin, glycerol esters of hydrogenated rosin, glycerol esters of polymerized rosin, pentaerythritol esters of natural and modified rosins including pentaerythritol esters of pale wood rosin, pentaerythritol esters of hydrogenated rosin, pentaerythritol esters of tall oil rosin, and phenolic-modified pentaerythritol esters of rosin.

Useful tackifying agents are commercially available under a variety of trade designations including, e.g., hydrocarbon tackifying resins under the ESCOREZ series of trade designations, from Exxon Mobil Chemical Company (Houston, Tex.) including ESCOREZ 5400 (1% aromatic content), ESCOREZ 5415, ESCOREZ 5600 (9.8% aromatic content), ESCOREZ 5690 (10% aromatic content), ESCOREZ 5615 (9.9% aromatic content), hydrocarbon tackifying resins under the EASTOTAC series of trade designations, from Eastman Chemical (Kingsport, Tenn.) including EASTOTAC H-100R and EASTOTAC H-100L, hydrocarbon C-5 derived tackifying resins under the WINGTACK series of trade designations, hydrocarbon C-5 derived tackifying resins from Cray Valley HSC (Exton, Pa.) including WINGTACK 86, WINGTACK EXTRA, and WINGTACK 95 and hydrocarbon C-5 derived tackifying resin, under the PICCOTAC and pure monomer aromatic tackifying resins under the KRISTALEX and PLASTOLYN series of trade designations from Eastman Chemical Company (Kingsport, Tenn.) including, e.g., PICCOTAC 8095, KRISTALEX 3100, PLASTOLYN 240 and PLASTOLYN 290.

The thermoplastic film can include at least one tackifying agent with aromatic content. The tackifying agent can have an aromatic content of greater than 5%, greater than 20%, greater than 50%, from about 5% to about 20% by weight, from about 7.5% to about 15% by weight, or even from 5% by weight to less than 10% by weight. The aromatic content is measured by Nuclear Magnetic Resonance (NMR) spectroscopy.

The thermoplastic film can include a tackifying agent with a softening point of less than 100° C., or even less than 95° C. The thermoplastic film can include a tackifying agent with an aromatic content of greater than 50% by weight, having a softening point of greater than about 100° C., greater than about 120° C., or even from about 105° C. to about 160° C.

### Plasticizer

The thermoplastic film can include plasticizer. Suitable plasticizers include, e.g., oil (e.g. naphthenic oils, paraffinic oils (e.g., cycloparaffin oils), mineral oils), phthalate esters, adipate esters, olefin oligomers (e.g., oligomers of polypropylene, polybutene, and hydrogenated polyisoprene), polybutenes, polyisoprene, hydrogenated polyisoprene, polybutadiene, benzoate esters, animal oil, plant oils (e.g. castor oil, soybean oil (e.g. high oleic soy oil)), derivatives of oils, glycerol esters of fatty acids, polyesters, polyethers, lactic acid derivatives and combinations thereof.

Useful commercially available plasticizers include CALSOL 5550, a naphthenic oil from Calumet Specialty Products Partners, LP (Indianapolis, Ind.), KAYDOL OIL mineral oil from Sonneborn (Tarrytown N.Y.) PARAPOL polybutene from Exxon Mobil Chemical Company (Houston, Tex.), OPPANOL polyisobutylene from BASF (Ludwigshafen, Germany), KRYSTOL 550 mineral oil from Petrochem Carless Limited (Surrey, England), PURETOL 35 and 15 both mineral oils from Petro Canada Lubricants Inc. (Mississauga, Ontario), PLENISH from Pioneer Dupont, and TPC 5230, polyisobutylene available from TPC Group (Houston, Tex.).

The thermoplastic film can include less than 30% by weight, less than 25% by weight, from about 5% by weight to about 25% by weight, or even from 10% by weight to about 20% by weight of a plasticizer.

### Additional Materials

The thermoplastic film can optionally include various additional materials including, e.g., additional polymers (lower MFR SBC's, olefins, etc.), waxes, stabilizers, antioxidants, adhesion promoters (e.g. functionalized materials (e.g. functionalized waxes, polymer, etc.)), ultraviolet light stabilizers, corrosion inhibitors, odor absorbers/neutralizers, colorants (e.g., pigments and dyes), fragrances, fillers (e.g. nano particles, clay, talc), surfactants, wetness indicators, superabsorbents, coextrusion coatings, processing aids (e.g. dusting agents) and combinations thereof.

Useful antioxidants include, pentaerythritol tetrakis[3,(3,5-di-tert-butyl-4-hydroxyphenyl)propionate], 2,2′-methylene bis(4-methyl-6-tort-butylphenol), phosphites including, e.g., tris-(p-nonylphenyl)-phosphite (TNPP) and bis(2,4-di-tert-butylphenyl)4,4′-diphenylene-diphosphonite, di-stearyl-3,3′-thiodipropionate (DSTDP), and combinations thereof. Useful antioxidants are commercially available under a variety of trade designations including, e.g., the IRGANOX series of trade designations including, e.g., IRGANOX 1010, IRGANOX 565, and IRGANOX 1076 hindered phenolic antioxidants and IRGAFOS 168 phosphite antioxidant, all of which are available from BASF Corporation (Florham Park, N.J.), and ETHYL 702 4,4′-methylene bis(2,6-di-tert-butylphenol). When present, the thermoplastic film preferably includes from about 0.1% by weight to about 2% by weight antioxidant.

### Methods of Making

The thermoplastic film can be made by heating and extruding (e.g. cast film, blown film, etc.). The molten film can be extruded directly on to a substrate.

Alternatively, the molten film can be extruded into a self supporting film. After extrusion, the film can be cooled e.g. by passing through a water bath. The film can further be dusted or skinned (i.e. a second layer of material (e.g. polyolefin polymer) is coated or simultaneously extruded on at least the top and the bottom surfaces of the film) in order to make the film less tacky and easier to handle and wind.

### Elastic Composite

The elastic composite can include a first substrate and the thermoplastic film. The thermoplastic film can be layered on top of the first substrate to impart elasticity to the first substrate. The term “layered on top” includes any level of penetration into the first substrate. In some cases, it may be desirable to have the thermoplastic film penetrate into the substrate. in other cases, it may be desirable to have the thermoplastic film penetrate only very slightly or not at all into the substrate.

The thermoplastic film can be adhered to the substrate using techniques known in the art, e.g. by applying heat, by ultrasonic bonding, by a separate adhesive (e.g. hot melt adhesive), etc. Alternatively, the thermoplastic film can be applied in a molten state, adhering to the substrate as it is applied.

The elastic composite can include a first substrate, a second substrate and a thermoplastic film. The thermoplastic film is present between the first and second substrate, providing elasticity to the composite.

At least one of the substrates is selected from the group consisting of nonwoven, polymer film and textiles (i.e. fabric formed by weaving (e.g. woven fabric (e.g. cotton, canvas, etc.), knitting, crocheting, knotting or felting).

Any nonwoven can be used. The nonwoven can be an elastic nonwoven (e.g. core and shell type). The nonwoven can contain fibers made from one or more polymers (e.g. PET (polyethylene terephthalate), PBT (polybutylene terephthalate), nylon, polypropylene and polyethylene), one or more natural fibers (e.g. rayon cellulose, cotton cellulose, hemp and viscose) or combinations thereof. The nonwoven can be formed by a number of different methods, including e.g. airlaid, wetlaid, spunbound or meltblown. The fibers can be carded (e.g. run through a comb) so that they are oriented in a particular direction. The webs can be bonded together in any manner including e.g. hydroentangled, chemical bonded, needle punched or thermally bonded.

Any polymer film can be used. The polymer film can be selected from the group consisting of polyethylenes (e.g. low density polyethylene, linear low density polyethylene, high density polyethylene), polypropylenes, polyethylene copolymers, polypropylene copolymers, polyester (e.g. PET), polyurethane, ethylene-vinyl acetate, ethylene-methacrylic acid ionomers, ethylene-vinyl-alcohols, polycarbonates, polyamides e.g. Nylon-6 and Nylon-6,6, polyimides, polyvinyl chloride, polyvinylidene chloride, cellulosics, polystyrene, epoxy and polymer composites (e.g., composites of a polymer and metal, cellulose, glass, polymer, and combinations thereof)

The first and second substrates can be nonwoven. The nonwoven can have a basis weight of less than 40 grams per square meter (gsm), less than 35 gsm, or even less than about 30 gsm. The nonwoven can be extensible to greater than 100% in the cross-web direction.

Various pre treatments (e.g. pleating and creping) and various post treatments, such as treatment with grooved rolls i.e. activation can be used to adjust the mechanical properties (e.g. extensibility) of the composite.

The thermoplastic film can be applied to the first and or second substrate using a variety of application methods including slot coating, non-contact coating and extrusion.

The thermoplastic film can be applied to one substrate.

Alternatively, the thermoplastic film can be applied to the first substrate and then contacted by the second substrate to form the composite. Pressure or tension can be used to aid in forming the bonded composite. The composite can be formed within the manufacturing process. Alternatively, the composite is formed prior to the the manufacturing process.

### Article

The thermoplastic film and elastic composites of this invention can be incorporated into any suitable article including personal care garments, medical garments, industrial worker garments and wearable electronics.

The elastic composite of this invention is useful in a variety of applications and constructions to improve comfort and fit including e.g., disposable absorbent articles including, e.g., diapers, training paints, swim wear, absorbent underpants, adult incontinence products, feminine hygiene products (e.g. sanitary napkins and liners), medical dressings (e.g., wound care products and bandages), surgical pads, medical gowns, caps, gloves, drapes, face masks, laboratory coats, coveralls, meat-packing products, furniture covers (e.g. car seat covers) and components of absorbent articles including, e.g., an absorbent element, absorbent cores, impermeable layers (e.g., backsheets), tissue (e.g., wrapping tissue), acquisition layers and woven and nonwoven web layers (e.g., top sheets, absorbent tissue).

The elastic composite of this invention is useful for elasticizing many areas of disposable articles including leg cuffs, waist portions, belly bands, side panels and fastening tabs/ears. The elastic composite of this invention can further be use to elasticize any portion of the disposable article or even the entire disposable article.

### EXAMPLES

### Test Procedures

Test procedures used in the examples and throughout the specification, unless stated otherwise, include the following.

### Melt Flow Rate

Melt Flow Rate was run according to ASTM D 1238 at the conditions stated.

### Mechanical Hysteresis

Test films were prepared using a slot die applicator connected to a simple 3 zone, single screw extruder with the conditions as outlined in Table 2. The samples were slot coated onto Mylar release liner and nipped with another Mylar release liner. Test samples are prepared by cutting the films in cross-web direction and the machine-web direction as noted in the table, with 2.54 cm (1 in) in width and at least 8.9 cm (3.5 in) in length. Grip separation is set to 5.1 cm (2 in).

The test is conducted at least 24 hours after the films were prepared. The strips prepared are extended to a 50% strain and then retracted to their original dimension. Subsequently the specimen goes through a second extension-retraction cycle with the same deformation. The cross head speed is set to 50.8 cm/min (20 in/min) during the extension portion of the cycle and 101.6 cm/min (40 in/min) during the retraction portion of the cycle. There is no holding time between extension and retraction. The samples are run on an INSTRON type-test instrument with at least 3 replicates. The energy loss during each hysteresis cycle and the total hysteresis cycle energy are calculated. The mechanical hysteresis is the ratio of the energy loss during the hysteresis cycle to the total hysteresis energy. Load at 50% extension (N) is also recorded. Similarly, this test is performed at the peak extension of 100% elongation.

### Tensile Test

The films were prepared as described above. The test samples were prepared by cutting the films in the cross-web direction, with an ASTM type V dogbone punch mold. Grip separation is set to 2.54 cm (1 in). The test is conducted at least 24 hours after the films were prepared. The strips prepared were tested for tensile properties in compliance with the ASTM method for thin elastic films. The specimens were extended at a cross head speed of 12 in/min until break or until maximum extension of the machine is reached (950% elongation), whichever event happens first. The samples are run on an INSTRON type-test instrument with at least 3 replicates. The stress at maximum load, the tensile strain at break, and the energy at break are all recorded.

### Polymer Descriptions

VISTAMAXX 6202 is a propylene ethylene copolymer commercially available from ExxonMobil Chemical (Houston, Tex.).

ENGAGE 8200 is an ethylene octene copolymer commercially available from DowDupont Chemical Company (Midland, Mich.).

POLYMER 1 is a <1 weight % diblock, SIS tri-block co-polymer containing 30% by weight styrene and having an ASTM D 1238 MFR (190° C., 2.16 kg)=23 g/10-min).

TABLE ONE

| | Control 1 | Control 2 | |
|---|---|---|---|
| | VISTAMAXX | ENGAGE | Example 1 |
| | 6202 | 8200 | POLYMER 1 |
| MFR (190° C., 2.16 kg) | 9.1 | 5 | 23 |
| Tensile Properties | | | |
| (Cross Direction) | | | |
| Stress at Max Load (MPa) | 1.39 | 3.00 | 2.07 |
| Tensile Strain at Break (%) | 968.6 | 931.5 | 971.1 |
| Energy at Break (J) | 0.24 | 0.38 | 0.26 |

TABLE TWO

| | Control 1 | Control 2 | |
|---|---|---|---|
| | VISTAMAXX | ENGAGE | Example 1 |
| | 6202 | 8200 | POLYMER 1 |
| Run Conditions | | | |
| Extruder Heater | 218 | 218 | 204 |
| Temp (° C.) | | | |
| Melt Temp (° C.) | 200 | 202 | 191 |
| Motor Speed (rpm) | 20 | 20 | 20 |
| Hose/Applicator | 204/218 | 204/218 | 204 |
| Temp (° C.) | | | |
| Run Speed (meters/minute) | 7.31 | 7.62 | 8.53 |
| Coat Weight (gsm) | 47 | 47 | 46.5 |
| Mechanical Hysteresis | | | |
| (Cross Direction) | | | |
| 50% strain | | | |
| 1st cycle | 0.328 | 0.280 | 0.279 |
| 2nd cycle | 0.169 | 0.154 | 0.175 |
| 100% strain | | | |
| 1st cycle | 0.419 | 0.478 | 0.313 |
| 2nd cycle | .207 | .269 | .192 |
| Mechanical Hysteresis | | | |
| (Machine Direction) | | | |
| 50% strain | | | |
| 1st cycle | 0.324 | 0.251 | 0.494 |
| 2nd cycle | 0.161 | 0.125 | 0.177 |
| 100% strain | | | |
| 1st cycle | 0.424 | 0.415 | 0.496 |
| 2nd cycle | 0.192 | 0.201 | 0.166 |

What is claimed is

1. A thermoplastic film comprising greater than 70% by weight of a styrene block copolymer having an unsaturated mid block, diblock content of no greater than about 10% by weight and a MFR of from about 12 to about 200 ASTM D 1238 (190° C., 2.16-kg).

2. The thermoplastic film of claim 1 wherein the thermoplastic film has a cross direction mechanical hysteresis according to the Mechanical Hysteresis test method of from 0 to about 0.70.

3. The thermoplastic film of claim 1 wherein the thermoplastic film is elastic.

4. The thermoplastic film of claim 1 wherein the thermoplastic film comprises greater than 80% by weight of the styrene block copolymer.

5. The thermoplastic film of claim 1 wherein the thermoplastic film consists essentially of the styrene block copolymer.

6. The thermoplastic film of claim 1 wherein the styrene block copolymer has an average styrene content of from about 25% by weight to about 40% by weight.

7. The thermoplastic film of claim 1 wherein the styrene block copolymer has an ASTM D 1238 (190° C., 2.16 kg) melt flow rate of from about 15 to about 100.

8. The thermoplastic film of claim 1 wherein the styrene block copolymer is a styrene-isoprene-styrene block copolymer.

9. The thermoplastic film of claim 1, wherein the thermoplastic film is free of a tackifying agent.

10. An elastic composite comprising:
a first substrate; and
the thermoplastic film of claim 1 layered on top of the first substrate.

11. The elastic composite of claim 10 wherein the first substrate is nonwoven.

12. The elastic composite of claim 10 wherein the thermoplastic film is applied to the substrate while molten.

13. The elastic composite of claim 12 wherein the thermoplastic film is applied using an applicator method selected from the group consisting of slot and non-contact coating.

14. A disposable article comprising the elastic composite of claim 10.

15. The disposable article of claim 14 selected from the group consisting of diaper, adult incontinence product, feminine hygiene product and medical bandage.

16. A method of forming an elastic composite comprising the steps of:
applying a molten thermoplastic film to one substrate, and
contacting the molten thermoplastic film to a second substrate to form an elastic composite, the thermoplastic film comprising greater than 70 % by weight of a styrene block copolymer having an unsaturated mid block, diblock content of no greater than about 10% by weight and a MFR of from about 12 to about 200 ASTM D 1238 (190° C., 2.16-kg).
